(19) United States
(12) Patent Application Publication
(10) Pub. No.: US 2014/0066756 A1
(43) Pub. Date: Mar. 6, 2014

(54) LOW COST MOLDED OPTICAL PROBE WITH ASTIGMATIC CORRECTION, FIBER PORT, LOW BACK REFLECTION, AND HIGHLY REPRODUCIBLE IN MANUFACTURING QUANTITIES

(71) Applicant: NinePoint Medical, Inc., Cambridge, MA (US)

(72) Inventors: Matthew A. Sinclair, Somerville, MA (US); Mason L. Riley, Halifax, MA (US); Narissa Y. Chang, Somerville, MA (US); Eman Namati, Arlington, MA (US)

(73) Assignee: NinePoint Medical, Inc., Cambridge, MA (US)

(21) Appl. No.: 14/011,191

(22) Filed: Aug. 27, 2013

Related U.S. Application Data
(60) Provisional application No. 61/696,616, filed on Sep. 4, 2012.

(51) Int. Cl. A61B 5/00

(52) U.S. Cl. CPC A61B 5/0077
USPC 600/427; 156/245; 264/1.24

(57) ABSTRACT

A low cost molded optical probe with astigmatic correction, fiber port, low back reflection, and highly reproducible in manufacturing quantities is provided. The molded optical probe, includes a fiber receiving portion defining a groove defined along a longitudinal axis for receiving an optical fiber; a spacer portion having a spacer portion surface non-orthogonal to the longitudinal axis of the groove, the spacer portion surface configured to cooperate with a distal end of the optical fiber; a prism portion positioned adjacent the spacer portion and having a prism surface non-parallel to the spacer portion surface and non-orthogonal to the longitudinal axis and configured to reflect light transmit through the optical fiber off perpendicular to the longitudinal axis; and a lens portion positioned adjacent the prism portion and having a lens surface configured to focus light received through the optical fiber.

### TECHNICAL FIELD

The present disclosure generally relates to medical devices, systems and methods for imaging in biomedical and other medical and non-medical applications, and more particularly, to optical probes for Optical Coherence Tomography (OCT) imaging.

### BACKGROUND

Various forms of imaging systems are used in healthcare to produce images of a patient. Often, an image of an internal cavity of a patient is required. These cavities can include areas of the digestive system or the respiratory system. When imaging tissue features of these systems, fiber optic endoscopy is often utilized.

One type of fiber optic endoscope is based on Optical Coherence Tomography (OCT) techniques. OCT provides structural information on tissue with high resolution. OCT can provide this information in real time and in a non-invasive manner. Many different lens types have been used to construct fiber optic endoscopes. These lenses include fiber lenses, ball lenses and GRadient INdex (GRIN) lenses. Lens materials can vary from glass to plastic to silicon.

As shown in FIG. 1, one type of OCT probe 10 for a fiber optic endoscope is comprised of an optical fiber 11 having a casing 11a, a fiber core 11b, a proximal end 12 and a distal end 13, a spacer 16 connected to the distal end of the optical fiber 11, a GRIN lens 14 connected to spacer 16, and a prism 15 connected to GRIN lens 14 and configured to deflect light into surrounding tissue T. Spacer 16 is positioned before the GRIN lens to modify the optical parameters. The separate components, i.e. fiber core 11b, GRIN lens 14, prism 15, and spacer 16, are typically connected by fusing the components together or using an epoxy to glue the components together. In total, this design requires 8 distinct and separate surfaces that light must travel through in a probe of this design.

Probe 10 is typically connected to a coherent light source 16 at proximal end 12 of optical fiber 11. Probe 10 is typically contained within a sheath S and a balloon B. Sheath S containing probe 10 is inserted into a cavity of a patient to image into tissue T surrounding probe 10. Sheath S protects probe 10 and tissue T from damage.

An optical probe must be specifically manufactured to conform to optical parameters required for a specific use. Esophageal imaging requires probes of specific design to properly image into surrounding tissue. Typical prior art probes do not provide the specific optical operating parameters required in esophageal imaging.

Often these prior art probes are expensive to manufacture due to the fine tolerances (often in the microns) required during the manufacturing process. In addition, conventional probes create astigmatic errors and aberrations due to even minor defects in the surfaces of the components. Any increase in the number of surfaces will increase the possibility of surface defects. Other problems associated with these conventional probes include back reflections off the multiple surfaces between the required components that can prevent the formation of an image or even destroy the components due to high heat generated by the back reflections. Any increase in the number of surfaces will increase the possibility of an increase in back reflection and other aberrations.

Problems with aberration and back reflection are typical in conventional probes. This typically occurs when mating and other surfaces are perpendicular to the light path. Conventional optical probes force this correction by maintaining tight tolerances to component design, while maintaining the perpendicular surfaces. This is usual in the designing of conventional optical probes because the assembly of perpendicular surfaces makes for easier manufacturing.

This disclosure describes improvements over these prior art technologies.

### SUMMARY

Accordingly, a low cost molded optical probe with astigmatic correction, fiber port, low back reflection, and highly reproducible in manufacturing quantities is provided.

A molded optical probe according to the present disclosure, includes a fiber receiving portion defining a groove defined along a longitudinal axis for receiving an optical fiber; a spacer portion having a spacer portion surface non-orthogonal to the longitudinal axis of the groove, the spacer portion surface configured to cooperate with a distal end of the optical fiber; a prism portion positioned adjacent the spacer portion and having a prism surface non-parallel to the spacer portion surface and non-orthogonal to the longitudinal axis and configured to reflect light transmit through the optical fiber off perpendicular to the longitudinal axis; and a lens portion positioned adjacent the prism portion and having a lens surface configured to focus light received through the optical fiber.

A molded optical probe with astigmatic correction, fiber port, and low back reflection according to the present disclosure includes a fiber receiving portion defining a groove defined along a longitudinal axis for receiving an optical fiber and an outer insulator, including a first section configured to receive the outer insulator containing the optical fiber; and a second section configured to receive the optical fiber; a spacer portion having a spacer portion surface non-orthogonal to the longitudinal axis of the groove, the spacer portion surface configured to cooperate with a distal end of the optical fiber; a prism portion positioned adjacent the spacer portion and having a prism surface non-parallel to the spacer portion surface and non-orthogonal to the longitudinal axis and configured to reflect light transmit through the optical fiber off perpendicular to the longitudinal axis; and a lens portion positioned adjacent the prism portion and having a lens surface configured to focus light received through the optical fiber onto a surface, wherein the molded optical probe is monolithic.

A method for manufacturing a molded optical probe according to the present disclosure includes molding the optical probe; stripping the outer insulator to expose the optical fiber; spacing the distal end of the optical fiber from the spacer portion surface at a set distance to adjust for optical tolerances; and attaching the optical fiber and the insulator to the molded optical probe using an optical adhesive having a specified index of refraction.

A method for manufacturing a molded optical probe including a fiber receiving portion, a spacer portion having a spacer portion surface non-orthogonal to the longitudinal axis of the groove, the spacer portion surface configured to cooperate with a distal end of an optical fiber, a prism portion positioned adjacent the spacer portion and having a prism surface non-parallel to the spacer portion surface and non-orthogonal to the longitudinal axis and configured to reflect light transmit through the optical fiber off perpendicular to the longitudinal axis, and a lens portion positioned adjacent the prism portion and having a lens surface configured to focus light received through the optical fiber, according to the present disclosure includes cleaving the distal end of the optical fiber; positioning the optical fiber into an injection mold; and injection molding the molded optical probe about the optical fiber.

### BRIEF DESCRIPTION OF THE DRAWINGS

The present disclosure will become more readily apparent from the specific description accompanied by the following drawings, in which:

FIG. 1 is a diagram illustrating a conventional optical probe having a gradient index lens;

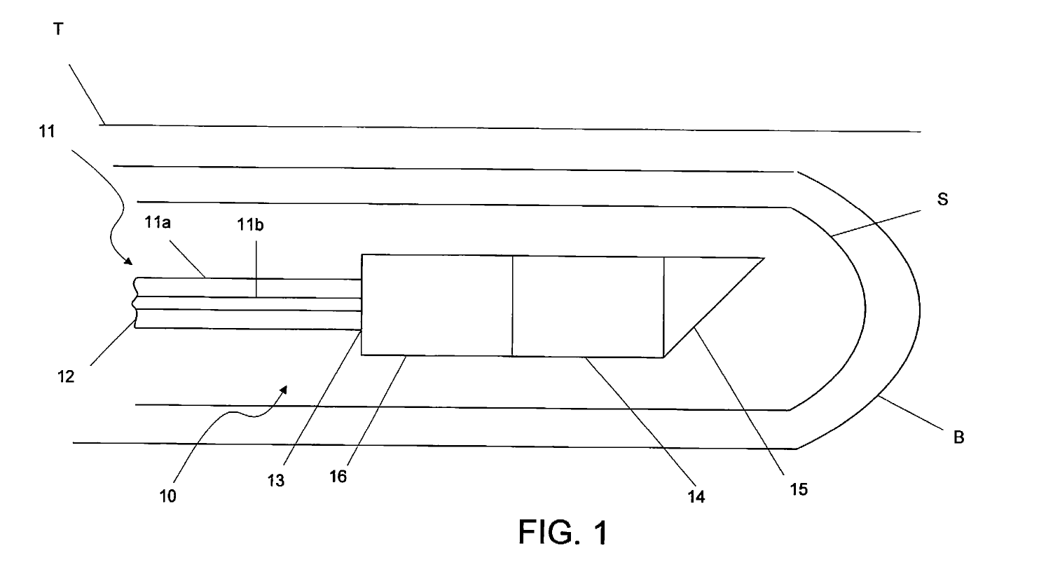

FIG. 2 is a diagram illustrating various operating parameters of an optical probe;

FIG. 3 is a perspective view of the optical probe according to the present disclosure;

FIG. 4 is a perspective view of the optical probe according to the present disclosure;

FIG. 5 is a perspective view of the optical probe including a fiber optic cable according to the present disclosure;

FIGS. 6A-6F are plan views of the optical probe according to the present disclosure; and

FIG. 7 is a diagram illustrating optical probe specifications according to an embodiment of the present disclosure.

Like reference numerals indicate similar parts throughout the figures.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure.

Reference will now be made in detail to the exemplary embodiments of the present disclosure, which are illustrated in the accompanying figures.

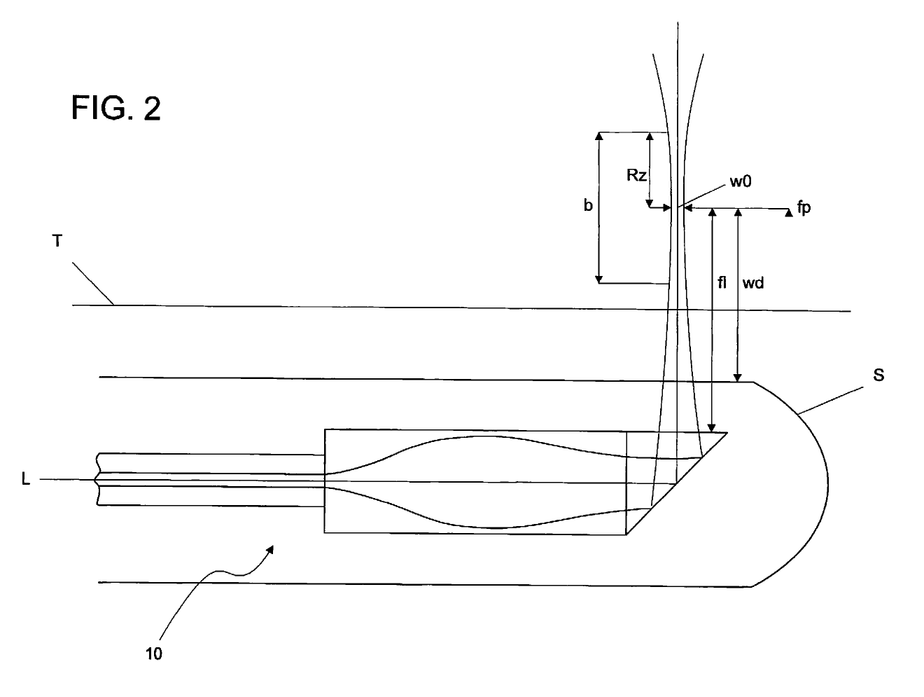

Referring to FIG. 2, proper imaging into tissue using an OCT probe required strict compliance to probe specifications in order to precisely set the optical parameters. These parameters can include the Rayleigh Range Rz, the confocal parameter b, the waist w0, the focal point fp, the focal length fl, and the working distance wd. The term “beam waist” or “waist” as used herein refers to a location along a beam where the beam radius is a local minimum and where the wavefront of the beam is planar over a substantial length (i.e., a confocal length). The term “working distance” as used herein means the distance between the outer surface of the sheath and the focal point fp.

As stated above, an optical probe must be specifically manufactured to conform to these optical parameters. Esophageal imaging requires probes of specific design to properly image into surrounding tissue T. When using an optical probe for esophageal imaging, a long working distance with large confocal parameter is required. Generally in esophageal imaging the working distances from the center of the optical probe radially outward to the tissue ranges from 7 mm to 12.5 mm. The optic itself can be 1 mm in diameter, with a protective cover (not shown) in sheath S, and with balloon B on top, while still fitting through a 2.8 mm channel in an endoscope. With no tight turns required during the imaging of the esophagus (compared, for example, to the biliary system, digestive system or circulatory system), an optical probe can be as long as 12.5 mm in length without a interfering with surrounding tissue T. In attempts to manufacture an optical probe that conforms to these parameters, several designs have been utilized.

One design utilizes a ball lens. A ball lens is costly to manufacture with little control and correction over aberrations caused by sheaths covering the optical probe. Another design uses a GRadient INdex (GRIN) lens. Unfortunately, the GRIN lens is also costly to manufacture with extremely tight tolerances due to the fast gradient index change and requires an additional compensator, usually a cylindrical right angle prism, for the protective sheath.

Another design utilizes an outer balloon structure B to increase the working distance. The use of balloon B often deforms the surrounding tissue and also creates another layer of aberration that needs to be corrected. Although this design creates these problems, some procedures still require the use of balloon B encapsulated probe structure to achieve ideal imaging conditions.

In attempts to correct for aberration and adjust the optical parameters, the use of a spacer placed between the fiber optic and the lens has been attempted. The variance of the spacer is a most crucial factor for determining the waist and the working distance of a probe. As a spacer length increases, the waist becomes smaller and the working distance becomes shorter.

As stated above, an optical probe must be specifically manufactured to conform to the optical parameters required for a specific procedure and application. Esophageal imaging requires probes of specific design to properly image into surrounding tissue T. When using an optical probe for esophageal imaging, a long working distance with large confocal parameter is required. In prior attempts, the manufacturing tolerances are extremely tight and do not allow for the production of an optical probe that is simple to manufacture and conforms to the optical parameters required in esophageal imaging.

The present disclosure teaches an optical probe that conforms to the specific requirements of esophageal imaging. In particular, the optical probe described herein is a low cost molded optical probe with astigmatic correction, fiber port, low back reflection, and highly reproducible in manufacturing quantities.

The present disclosure differs from the conventional designs in that a molded optical probe is configured such that the design allows for a probe with aberration correction of the outer sheath and balloon that is inexpensive to manufacture, possesses much lower tolerances to the manufacturing specifications, and has a long working distance with a large confocal parameter.

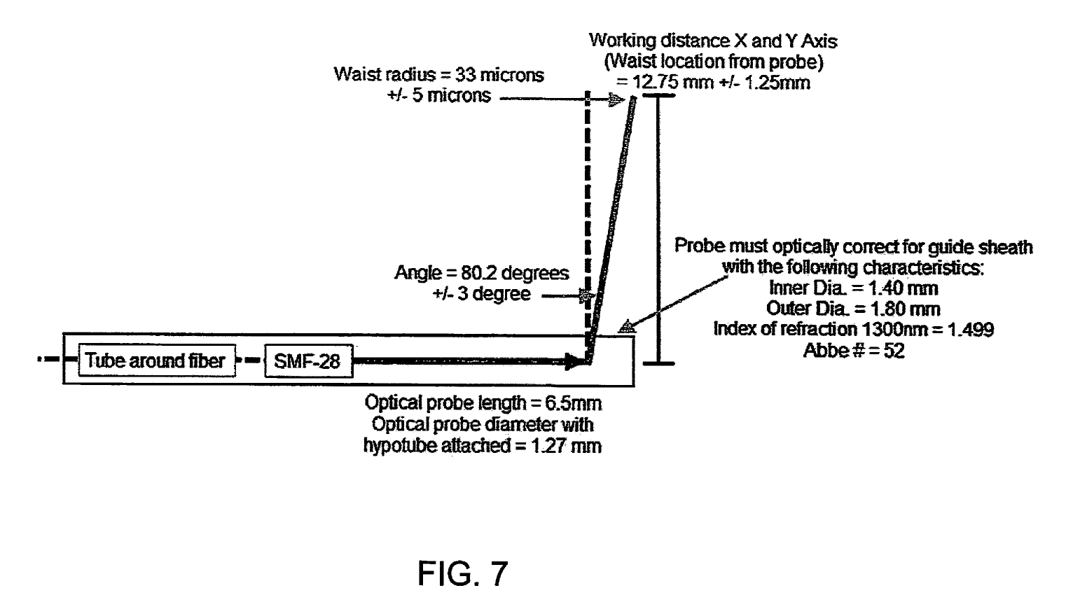

Optical probes for esophageal applications generally require the following specifications shown in Table 1, (SMF: single mode fiber) with additional probe specifications detailed in FIG. 7:

TABLE 1

| OVERALL SYSTEM | NOMINAL VALUE | | TOLERANCE | |
|---|---|---|---|---|
| Wavelength | 1300 | nm | +75/−75 | nm |
| Waist radius in X and Y axis (Gaussian | 33 | μm | +5/−5 | μm |
| 1/e\hat{ }2) | | | | |
| Waist location X and Y (radial from SMF | 12.75 | mm | +1.25/−1.25 | mm |
| ferrule optical axis) | | | | |
| Angle of deviation from SMF ferrule optical | 80.2 degrees | | +3/−3 degrees | |
| axis | | | | |
| Diameter of probe | 1 | mm | +0/−0.1 | mm |
| Probe length with prism | 6.5 | mm | +0.5/−0.5 | mm |
| Back reflection | ≦−60 dB | | | |
| Must correction for outer tube | Specifications on | | image | |
| Transmission | >75% w/prism | | | |
| Protective hypo-tube cover for optics | 1.07/1.27 | mm | +0.025/−0.025 | mm |

(inner/outer)

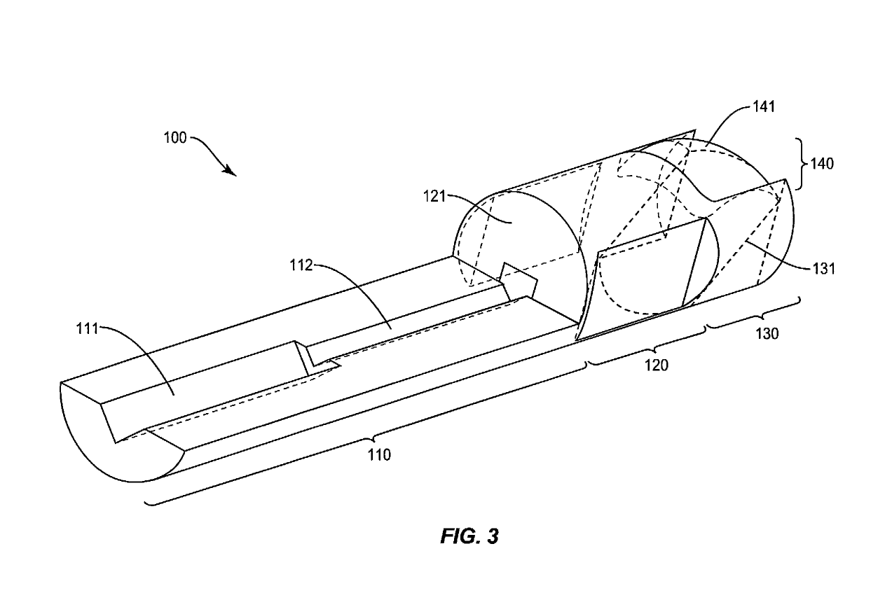

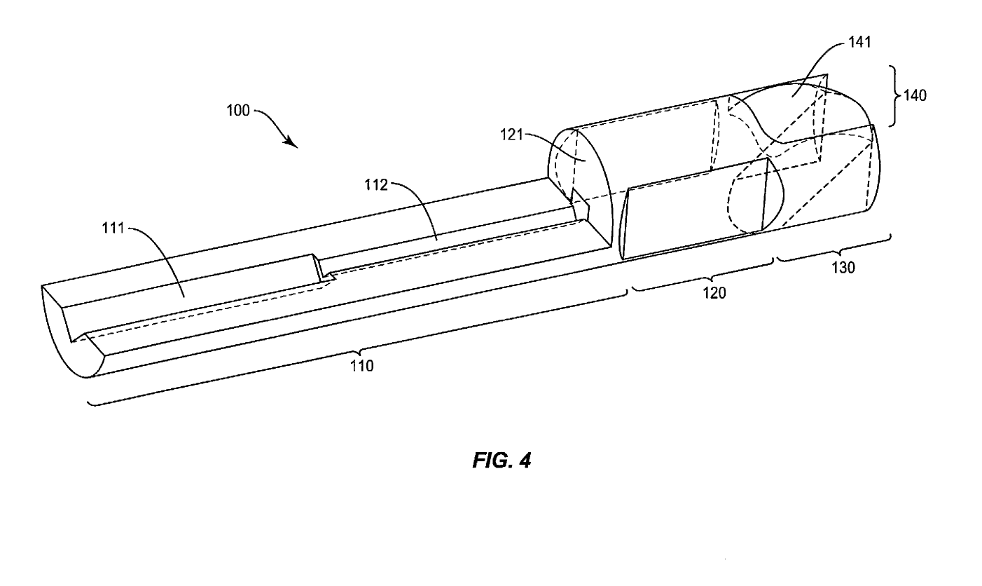

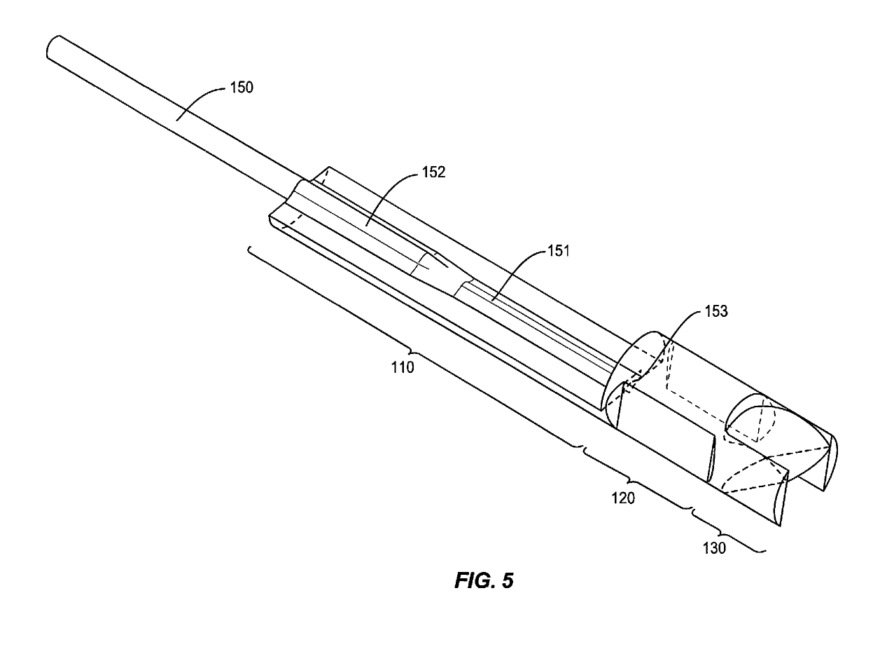

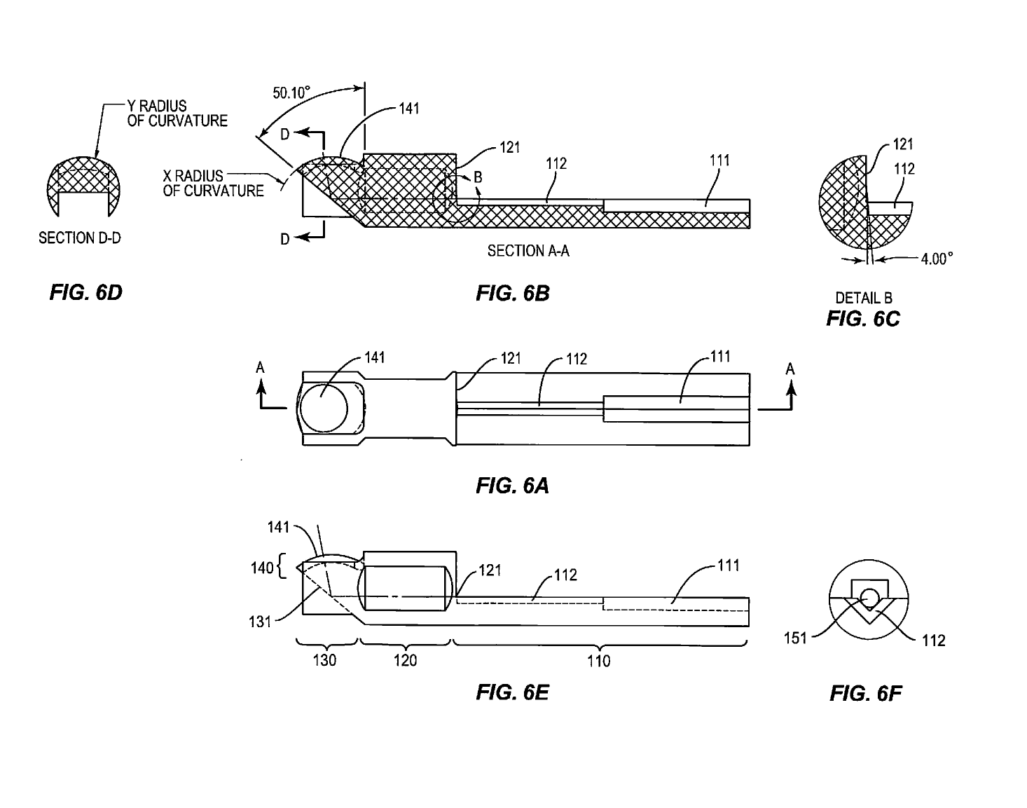

FIGS. 3-6F illustrate the optical probe in accordance with the present disclosure. Optical probe 100 is a molded device. Optical probe is preferable manufactured as a monolithic device, as will be discussed is further detail below. Optical probe 100 includes a fiber optic portion 110, a spacer portion 120, a prism portion 130 and a lens portion 140.

Fiber optic portion 110 includes at least one groove, but optimally includes 2 grooves 111 and 112. Grooves 111/112 are preferably configured in a “V” shape. Other configurations, for example, round, oval or squared, are contemplated. Groove 111 is configured to hold a fiber optic cable 150. Cable 150 includes an outer protective coating or insulator and an inner fiber optic 151. Fiber optic 151 ends at distal end 153. Groove 112 is configured to hold fiber optic 151. Thus, when a portion of the insulator is removed, fiber optic cable 150 can rest in groove 111 and fiber optic 151 can extend into groove 112. An optical epoxy or glue 152 is used to affix cable 150 and fiber optic 151 into grooves 111/112. Epoxy/glue 152 is generally selected to match the index of refraction of fiber optic 151 and spacer portion 120. Epoxy/glue 152 also acts as a component that can correct for minor defects on the surfaces of fiber optic 151 and probe 100. Preferably, distal end 153 is cleaved at an angle other than 90 degrees. The optimal cleave angle for the distal end of the fiber 153 is 8 degree+/−1.5 degrees, but can be in the range between 0 degrees and 10 degrees. The angle is based on back reflection and depending on how well the index of refraction of the glue matches the fiber, the angle of the fibers distal end may be reduced to 1 degree

Spacer portion 120 includes a surface 121. Spacer portion 120 is designed to transmit light from distal end 153 of fiber optic 151. Surface 121 is non-orthogonal to the longitudinal axis of grooves 111/112. The angle of surface 121 is optimal at 4 degree since the distal end of the fiber 153 is angled, ensuring minimal back reflection from the surface. This angle on surface 121 depends on the angle of the distal end of the fiber 153 and the angle on surface 121 will change accordingly to minimize reflections. The angle on surface 121 can be between −10 degrees and 10 degrees. The angle at which distal end 153 of fiber optic 151 is cleaved is preferably orthogonal to the angle at which surface 121 is manufactured. In another example, distal end 153 of fiber optic 151 is cleaved flat with surface 121 maintaining an angle. A fiber cleaver (not shown) cleaves fiber optic 151 at its preferred angle and is moved into grooves 111/112. Fiber optic cable 150 is placed within grooves 111/112 and epoxied into place.

The position of distal end 153 from surface 121 can be used to compensate for manufacturing defects in the probe, that is by changing the distance between distal end 153 and surface 121, the probe can be forced into specification tolerances. In one embodiment, distal end 153 may be placed a specific distance away from surface 121 under a microscope and then bonded in place. In another embodiment, active alignment is used as the optical characteristics are measured as distal end 153 is adjusted and then bonded down when the correct optical parameters are satisfied. In addition to modifying the space between distal end 153 and surface 121 to meet tolerances, another option is to change the mold itself by manufacturing the mold for surface 121 to include a compensating pin (not shown). The pin may be adjusted in the mold until the prescription is satisfied at which time the molding process can begin. The spacer portion 120 can be changed after a mold is created by changing the position of the pin in the mold with respect to grooves 111/112 and surface 121. This will change the distance of the grooves 111/112 slightly, but allows the fiber 150 to always be placed directly up against surface 121.

Any method may be used to compensate for imperfections in tooling. Thus, the probe can be manufactured with looser tolerances that can be compensated for by an accurate placement of optical fiber 151.

Prism portion 130 includes surface 131. Surface 131 is a reflective surface that is coated to maintain proper cleanliness of surface 131. The surface can be uncoated if cleanliness is enforced and light is reflected under total internal reflection (TIR). Surface 131 is angled to reflect the light at an angle off an axis perpendicular to the longitudinal axis. The optimal angle for the beam coming out of the probe is directly tied to how the probe is being used. In use, the probe will not have a perfect orientation to the tissue since the anatomy is often bent. The goal of the angle is to never be perpendicular to the tissue. The maximum angle the probe should see in reference to the tissue is 8 degrees; therefore, the optimal angle is 10 degrees angled off-axis from the tissue. Depending upon where the probe is used in the body, this angle may be as great as 80 degree and as low as 2 degrees.

Lens portion 140 includes surface 141. The optical axis of lens portion 140 is also tilted off-axis to further prevent any back reflections. The optimal ranges for surface 141 is 1.5 degrees to minimize back reflection since the beam angle will vary by 0.5 degrees during manufacturing. This angle redirects the Fresnel reflections and may be angled up to 8 degrees for a higher isolation from back reflections, but a reduced beam quality. Lens surface 141 can be at an angle off perpendicular to the longitudinal axis from −10 degrees to 10 degrees. Surface 141 being tilted is an alternative to coating the surface with an anti-reflection (AR) coating. AR coating materials such as magnesium fluoride (MgF2), silicon dioxide (SiO2), titanium dioxide (TiO2), and other metal oxides may be used to create a single or multilayer thin film of a given thickness to increase transmission by reducing Fresnel reflection. An AR coating may also be created by a surface texture as is done with the “moth eye” method. This method creates a sub-wavelength structure which breaks up the air/material interface and interrupts Fresnel reflections. If surface 141 exhibits too high a back reflection or is not tilted, then surface 141 may be AR coated. The AR coating will increase the power handling capability since Fresnel reflections will still occur on an uncoated surface. Reducing the Fresnel reflections even if they are not reflected back into the image will decrease the chance of high power damaging the probe material.

Surface 141 is a refractive surface capable of withstanding high power laser light. Surface 141 also is configured to correct for spherical aberrations. Surface 141 is a toroidal surface, preferably aspherical, configured having an X radius of curvature and a Y radius of curvature.

In order to address the aberration and back reflection issues, the optical probe 100 in accordance with the present disclosure has been designed such that none of the surfaces are perpendicular to the light path (unless AR coated, which adds cost to the manufacturing process). Any air to material or material to material transitions where there are different indexes of refraction, an AR coating or texturing of the surface to reduce Fresnel reflections may be used eliminating the need to have an angled surface on any of the given surfaces. Thus, distal end 153 of optical fiber 151, surface 121 of spacer portion 120, surface 131 of prism portion 130, and surface 141 of lens portion 140 are all off perpendicular to the light path L. Optimally, distal end 153 of optical fiber 151 is cleaved at 8 degrees, surface 121 of spacer portion 120 is angled at 4 degrees, reflective surface 131 of prism portion 130 is angled to direct the light approximately 80 degrees off the longitudinal axis, and surface 141 of lens portion 140 is tilted approximately 1.5 degrees off-axis. In addition, since the total number of surfaces is reduced to 4 (including distal end 153) this in and of itself reduces the chances of back reflection and distortions.

A method for manufacturing a molded optical probe according to the present disclosure is also provided. The method begins by molding the optical probe. This can be performed by an injection molding process or a stamp molding process. Next the outer insulator of the optical cable is stripped to expose the optical fiber. The stripped optical cable is placed into the groove and distal end 153 is spaced from spacer portion surface 121 at a set distance to adjust for the optical tolerances. Next the optical cable is attached to the molded optical probe using an optical adhesive having a specified index of refraction.

In another embodiment, the molded optical probe can be manufactured with the optical fiber attached during the molding process. In this embodiment, the grooves would not be provided as the probe is manufactured around the fiber during the manufacturing process. First, the distal end of the optical fiber is cleaved to specifications. The optical fiber can be with out without insulation (i.e. sheathing) during this process. If no insulation is provided at this stage, the optical fiber can be insulated after the probe is manufactured. Next, the optical fiber is positioned into an injection mold. Then, the molded optical probe is injection molded about the optical fiber.

The components of the system can be fabricated from materials suitable for medical applications, including glasses, plastics, polished optics, metals, synthetic polymers and ceramics, and/or their composites, depending on the particular application. For example, the components of the system, individually or collectively, can be fabricated from materials such as polycarbonates such as Lexan 1130, Lexan HPS26, Makrolon 3158, or Makrolon 2458, such as polyether Imides such as Ultem 1010, and/or such as polyethersulfones such as RTP 1400.

Various components of the system may be fabricated from material composites, including the above materials, to achieve various desired characteristics such as strength, rigidity, elasticity, flexibility, compliance, biomechanical performance, durability and radiolucency or imaging preference. The components of the system, individually or collectively, may also be fabricated from a heterogeneous material such as a combination of two or more of the above-described materials.

The present disclosure has been described herein in connection with an optical imaging system including an OCT probe. Other applications are contemplated.

Where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims.

What is claimed is:

1. A molded optical probe, comprising:
a fiber receiving portion defining a groove defined along a longitudinal axis for receiving an optical fiber;
a spacer portion having a spacer portion surface non-orthogonal to the longitudinal axis of the groove, the spacer portion surface configured to cooperate with a distal end of the optical fiber;
a prism portion positioned adjacent the spacer portion and having a prism surface non-parallel to the spacer portion surface and non-orthogonal to the longitudinal axis and configured to reflect light transmit through the optical fiber off perpendicular to the longitudinal axis; and
a lens portion positioned adjacent the prism portion and having a lens surface configured to focus light received through the optical fiber.

2. The molded optical probe of claim 1, wherein the fiber receiving portion, comprises:
a first section configured to receive an outer insulator containing the optical fiber; and
a second section configured to receive the optical fiber.

3. The molded optical probe of claim 1, wherein the molded optical probe is monolithic.

4. The molded optical probe of claim 1, wherein an optical axis of the lens portion is tilted 1.5 degrees off axis.

5. The molded optical probe of claim 1, wherein the lens surface is coated with an anti-reflective coating.

6. The molded optical probe of claim 1, wherein the spacer portion surface of the spacer portion and the distal end of the optical fiber are separated by a set distance to adjust for optical tolerances.

7. The molded optical probe of claim 6, wherein the spacer portion surface of the spacer portion includes a compensating pin in contact with the distal end of the optical fiber to separate the first surface from the distal end at the set distance.

8. The molded optical probe of claim 1, wherein the spacer portion surface of the spacer portion is manufactured at an angle between −10 degrees and 10 degrees.

9. The molded optical probe of claim 8, wherein the spacer portion surface of the spacer portion is manufactured at an angle of 4.00 degrees.

10. The molded optical probe of claim 1, wherein the distal end of the optical fiber is manufactured at an angle between 0 degrees and 10 degrees.

11. The molded optical probe of claim 10, wherein the distal end of the optical fiber is cleaved at an angle orthogonal to an angle of the spacer portion surface of the spacer portion.

12. The molded optical probe of claim 1, wherein the prism surface is at an angle off perpendicular to the longitudinal axis from 2 degrees to 80 degrees.

13. The molded optical probe of claim 12, wherein the prism surface is manufactured at an angle of 50.10 degrees.

14. The molded optical probe of claim 1, wherein the lens surface is at an angle off perpendicular to the longitudinal axis from −10 degrees to 10 degrees.

15. The molded optical probe of claim 14, wherein the lens surface is manufactured at an angle of 1.5 degrees off axis.

16. A molded optical probe with astigmatic correction, fiber port, and low back reflection, comprising:
a fiber receiving portion defining a groove defined along a longitudinal axis for receiving an optical fiber and an outer insulator, comprising:
a first section configured to receive the outer insulator containing the optical fiber; and
a second section configured to receive the optical fiber;
a spacer portion having a spacer portion surface non-orthogonal to the longitudinal axis of the groove, the spacer portion surface configured to cooperate with a distal end of the optical fiber;
a prism portion positioned adjacent the spacer portion and having a prism surface non-parallel to the spacer portion surface and non-orthogonal to the longitudinal axis and configured to reflect light transmit through the optical fiber off perpendicular to the longitudinal axis; and
a lens portion positioned adjacent the prism portion and having a lens surface configured to focus light received through the optical fiber onto a surface,
wherein the molded optical probe is monolithic.

17. The molded optical probe of claim 16, wherein the spacer portion surface of the spacer portion is manufactured at an angle between −10 degrees and 10 degrees.

18. The molded optical probe of claim 16, wherein the distal end of the optical fiber is manufactured at an angle between 0 degrees and 10 degrees.

19. The molded optical probe of claim 16, wherein the prism surface is at an angle off perpendicular to the longitudinal axis from 2 degrees to 80 degrees.

20. The molded optical probe of claim 16, wherein the lens surface is at an angle off perpendicular to the longitudinal axis from −10 degrees to 10 degrees.

21. A method for manufacturing a molded optical probe according to claim 1, comprising:
molding the optical probe;
stripping the outer insulator to expose the optical fiber;
spacing the distal end of the optical fiber from the spacer portion surface at a set distance to adjust for optical tolerances; and
attaching the optical fiber and the insulator to the molded optical probe using an optical adhesive having a specified index of refraction.

22. The method of claim 21, wherein the molding is performed by an injection molding process or a stamp molding process.

23. A method for manufacturing a molded optical probe including a fiber receiving portion, a spacer portion having a spacer portion surface non-orthogonal to the longitudinal axis of the fiber receiving portion, the spacer portion surface configured to cooperate with a distal end of an optical fiber, a prism portion positioned adjacent the spacer portion and having a prism surface non-parallel to the spacer portion surface and non-orthogonal to the longitudinal axis and configured to reflect light transmit through the optical fiber off perpendicular to the longitudinal axis, and a lens portion positioned adjacent the prism portion and having a lens surface configured to focus light received through the optical fiber, comprising:
cleaving the distal end of the optical fiber;
positioning the optical fiber into an injection mold; and
injection molding the molded optical probe about the optical fiber.
